(19) United States
(12) Patent Application Publication
(10) Pub. No.: US 2022/0320890 A1
(43) Pub. Date: Oct. 6, 2022

(54) WIRELESS POWER TRANSFER AND HEAT MITIGATION CIRCUIT FOR A RECHARGEABLE IMPLANTABLE PULSE GENERATOR

(71) Applicant: Advanced Neuromodulation Systems, Inc., Plano, TX (US)

(72) Inventors: Hongxuan Zhang, Austin, TX (US); Li Sun, Plano, TX (US); Luis Ortiz-Hernandez, Plano, TX (US)

(21) Appl. No.: 17/410,796

(22) Filed: Aug. 24, 2021

Related U.S. Application Data
(60) Provisional application No. 63/168,672, filed on Mar. 31, 2021.

(51) Int. Cl. H02J 7/00; H02J 50/12; H02J 50/20; A61N 1/378

(52) U.S. Cl. CPC H02J 7/007194; H02J 50/12; H02J 50/20; H02J 7/00711; H02J 7/00032; A61N 1/3787

(57) ABSTRACT

An implantable medical device (IMD) is configured to provide stimulation therapy to a patient. The IMD includes a rechargeable battery, pulse generating circuitry powered by the rechargeable battery and an inductive coupling element including at least one inductor operative to accept radio frequency (RF) power from an external charger and generate a charging voltage. A temperature sensor is configured to measure a temperature of at least a part of the IMD and output measured temperature data. A waveform generating circuit for converting measured temperature data to a waveform signal for controlling a recharging current for the rechargeable battery. The inductive coupling element is configured to communicate the waveform signal to the external charger.

### PRIORITY

This application claims priority to U.S. Provisional Application No. 63/168,672, filed Mar. 31, 2021, the entire contents of which are hereby incorporated by reference in its entirety.

### TECHNICAL FIELD

The present disclosure generally relates generally to implantable medical devices, and more particularly to a system and method for controlling charging energy delivered to an implantable medical device using wireless power transfer.

### BACKGROUND

Neurostimulation systems are devices that generate electrical pulses and deliver the pulses to nerve tissue to treat a variety of disorders. Spinal cord stimulation (SCS) is an example of neurostimulation in which electrical pulses are delivered to nerve tissue in the spine for the purpose of chronic pain control. Other examples include deep brain stimulation, cortical stimulation, cochlear nerve stimulation, peripheral nerve stimulation, vagal nerve stimulation, sacral nerve stimulation, and the like.

In addition to neurostimulation (NS) systems, numerous other medical devices exist today, including but not limited to electrocardiographs (ECGs), electroencephalographs (EEGs), squid magnetometers, implantable pacemakers, implantable cardioverter-defibrillators (ICOs), electrophysiology (EP) mapping and radio frequency (RF) ablation systems, and the like, that may be implanted within a patient for facilitating therapy and/or diagnostics.

In general, implantable medical devices (“IMDs”) are configured to be implanted within patient anatomy and commonly employ one or more electrical leads with electrodes that either receive or deliver voltage, current or other electromagnetic pulses from or to an organ or tissue for diagnostic or therapeutic purposes.

In order to provide consistent therapy and reliable operation over a substantial duration of time, IMDs are often provided with one or more rechargeable batteries that may be charged and recharged to store energy, which may supply power to the rest of the IMD circuitry and associated lead systems.

Because IMDs are implanted within patients, the IMDs are typically charged by an external charger that transmits energy wirelessly into the IMDs, such as through radio frequency (RF) signals. It is desirable that an IMD is generally charged as quickly and safely as possible within certain ranges depending upon the therapy application. However, if charging energy is input into the IMD too quickly and/or without proper regulation, the temperature of the IMD may increase to dangerous or uncomfortable levels causing tissue damage and other deleterious effects. It is further desired that wireless energy transfer between the external charger and the IMD's charging circuitry be performed as efficiently as possible.

Accordingly, there is a need to provide rapid, efficient and safe battery charging capabilities for IMDs.

### SUMMARY

In one embodiment, an implantable medical device (IMD) is configured to provide stimulation therapy to a patient, the IMD includes a rechargeable battery; pulse generating circuitry powered by the rechargeable battery; an inductive coupling element including at least one inductor operative to accept radio frequency (RF) power from an external charger and generate a charging voltage, a temperature sensor configured to measure a temperature of at least a part of the IMD and output measured temperature data; a waveform generating circuit for converting measured temperature data to a waveform signal for controlling a recharging current for the rechargeable battery; and wherein the inductive coupling element is configured to communicate the waveform signal to the external charger.

In another embodiment, a charging system for an implantable medical device is disclosed. The system includes: an external charging unit configured to provide radio frequency (RF) power; and an IMD comprising: a rechargeable battery; pulse generating circuitry powered by the rechargeable battery; an inductive coupling element including at least one inductor operative to accept the radio frequency (RF) power from the external charger and generate a charging voltage, a temperature sensor configured to measure a temperature of at least a part of the IMD and output measured temperature data; a waveform generating circuit for converting measured temperature data to a waveform signal for controlling a recharging current for the rechargeable battery; and wherein the inductive coupling element is configured to communicate the waveform signal to the external charger, and the external charger provides the RF power based upon the waveform signal.

In yet another embodiment, a method of charging an implantable medical device (IMD) is disclosed. The IMD includes a rechargeable battery, an inductive coupling element including at least one inductor operative to accept radio frequency (RF) power from an external charger and generate a charging current. The method includes: measuring, using a temperature sensor housed within the IMD, a temperature of at least a part of the IMD and outputting measured temperature data; communicate the outputted measured temperature data to a waveform generating circuit within the IMD, and converting the measured temperature data to a waveform signal for controlling a recharging current for the rechargeable battery; transmitting the waveform signal to the external charger, and outputting RF power from the external charger based upon the waveform signal.

Additional/alternative features, variations and/or advantages of the embodiments will be apparent in view of the following description and accompanying Figures.

### BRIEF DESCRIPTION OF THE DRAWINGS

Embodiments of the present disclosure are illustrated by way of example, and not by way of limitation, in the Figures of the accompanying drawings in which like references indicate similar elements. It should be noted that different references to “an” or “one” embodiment in this disclosure are not necessarily to the same embodiment, and such references may mean at least one. Further, when a particular feature, structure, or characteristic is described in connection with an embodiment, it is submitted that it is within the knowledge of one skilled in the art to effectuate such feature, structure, or characteristic in connection with other embodiments whether or not explicitly described.

The accompanying drawings are incorporated into and form a part of the specification to illustrate one or more exemplary embodiments of the present disclosure. Various advantages and features of the disclosure will be understood from the following Detailed Description taken in connection with the appended claims and with reference to the attached drawing Figures in which:

FIG. 1 depicts block diagrams of an external charging system and an implantable medical device (IMD) having wireless power transfer circuitry according to an embodiment;

FIG. 2 depicts a block diagram illustrating additional details of charge control and communications circuitry of an example IMD according to an embodiment;

FIG. 3 is a block diagram of a wireless power transfer system for purposes of an example embodiment of the present invention;

FIG. 4 is a circuit diagram of a frontend portion of a rechargeable IMD/IPG for facilitating wireless power transfer according to an embodiment of the present invention;

FIG. 5 depicts a flowchart of blocks, steps and/or acts that may be (re)combined in one or more arrangements with or without additional flowcharts of the present disclosure for facilitating charging operations according to some embodiments of the present disclosure;

FIG. 6 is a block diagram illustrating an external charging system and an implantable medical device (IMD) having wireless power transfer circuitry in use according to an embodiment;

FIG. 7 depicts an example IMD/IPG having a header portion and a body portion wherein an embodiment of the present disclosure may be practiced;

FIG. 8 illustrates a block diagram of IMD circuitry according to an embodiment of the present disclosure,

FIG. 9 depicts an IMD charging system having wireless transfer circuitry according to an example embodiment of the present disclosure, and

FIG. 10 depicts exemplary waveforms for charging control according to embodiments of the present disclosure.

### DETAILED DESCRIPTION

In the description herein for embodiments of the present disclosure, numerous specific details are provided, such as examples of circuits, devices, components, and/or methods, etc., to provide a thorough understanding of embodiments of the present disclosure. One skilled in the relevant art will recognize, however, that an embodiment of the disclosure can be practiced without one or more of the specific details, or with other apparatuses, systems, assemblies, methods, components, materials, parts, and/or the like. In other instances, well-known structures, materials, or operations are not specifically shown or described in detail to avoid obscuring aspects of embodiments of the present disclosure. Accordingly, it will be appreciated by one skilled in the art that the embodiments of the present disclosure may be practiced without such specific components. It should be further recognized that those of ordinary skill in the art, with the aid of the Detailed Description set forth herein and taking reference to the accompanying drawings, will be able to make and use one or more embodiments without undue experimentation.

Additionally, terms such as “coupled” and “connected,” along with their derivatives, may be used in the following description, claims, or both. It should be understood that these terms are not necessarily intended as synonyms for each other. “Coupled” may be used to indicate that two or more elements, which may or may not be in direct physical or electrical contact with each other, co-operate or interact with each other. “Connected” may be used to indicate the establishment of communication, i.e., a communicative relationship, between two or more elements that are coupled with each other. Further, in one or more example embodiments set forth herein, generally speaking, an electrical element, component or module may be configured to perform a function if the element may be programmed for performing or otherwise structurally arranged to perform that function.

Some embodiments described herein may be particularly set forth with respect to an implantable pulse generator (IPG) configured for generating electrical stimulation for application to a desired area of a body or tissue based on a suitable stimulation therapy application, such as a spinal cord stimulation (SCS) system. However, it should be understood that example wireless power transfer circuitry and methods of operation disclosed herein are not limited thereto, but have broad applicability, including but not limited to different types of implantable devices such as neuromuscular stimulators and sensors, dorsal root ganglion (DRG) stimulators, deep brain stimulator (DBS) devices, cochlear stimulators, retinal implanters, drug delivery systems, muscle stimulators, tissue stimulators, cardiac stimulators, gastric stimulators, and the like, including other bioelectrical sensors and sensing systems, which may be broadly referred to as “biostimulation” applications and/or implantable medical devices (IMDs) for purposes of the present disclosure. Moreover, example circuitry and methods of operation disclosed herein are not limited to use with respect to an IPG or any particular form of IPG or IMD. For example, some embodiments may be implemented with respect to a fully implantable pulse generator, a radio frequency (RF) pulse generator, an external pulse generator, a micro-implantable pulse generator, inter alia.

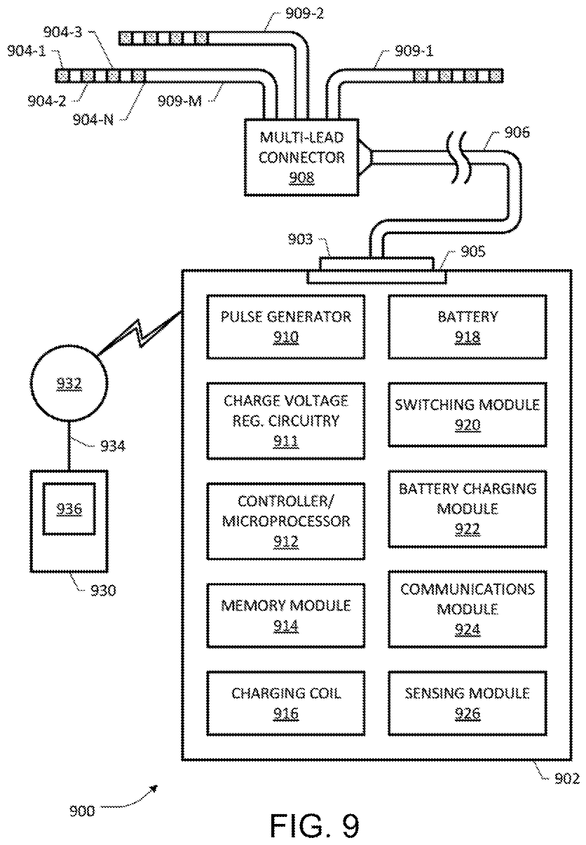

Referring to FIG. 9 in particular, depicted therein is a biostimulation system 900 wherein one or more embodiments the present disclosure may be practiced in association with an IPG/IMD for achieving optimized wireless power transfer from an external charging system according to the teachings herein. By way of illustration, system 900 may be adapted to stimulate spinal cord tissue, peripheral nerve tissue, deep brain tissue, ORG tissue, cortical tissue, cardiac tissue, digestive tissue, pelvic floor tissue, or any other suitable biological tissue of interest within a patient's body, as noted above. System 900 comprises IMD 902 having a pulse generator portion that is adapted to include or otherwise interoperate with (re)chargeable battery circuitry for generating suitable stimulation pulses having adjustable target voltages that may be selectively applied for purposes of therapy. As will be set forth below in additional detail hereinbelow, IMD 902 may be implemented in one example embodiment as having a metallic housing or can that encloses a controller/processing block or module 912, pulse generating circuitry 910, charging voltage regulation module 911, a charging coil 916, a battery 918, a far-field and/or near field communication block or module 924, battery charging circuitry 922, switching circuitry 920, sensing circuitry 926, one or more memory modules 914, and the like.

Controller/processor module 912 includes a microcontroller or other suitable processor, such as a processor specifically programmed or configured for controlling the various other components of IMD 902. Software/firmware code may be stored in memory 914, which may be integrated with the controller/processor module 912, and/or other suitable application-specific storage components (not particularly shown in this FIG.) for execution by the microcontroller or processor 912 and/or other programmable logic blocks to control the various components of IMD 902 for purposes of an embodiment of the present patent disclosure.

In one arrangement, IMD 902 may be configured to couple to one or more stimulation leads 909-1 to 909-M using an implantable multi-lead connector 908 operative to receive corresponding stimulation leads 909-1 to 909-M at their respective proximal ends for securely engaging and providing electrical connectivity with respect to each stimulation lead's distal end having a plurality of stimulation electrodes. By way of illustration, stimulation lead 909-M is exemplified with stimulation electrodes 904-1 to 904-N, which may be implanted near or adjacent to the patient's target tissue. Stimulation leads 909-1 to 909-M may comprise percutaneous leads, paddle leads, etc., wherein the electrodes may comprise ring electrodes, segmented or split electrodes, planar electrodes, and the like, that may be energized by the pulse generating circuitry 910 according to applicable therapy protocols/regimes. Preferably, a single lead cable 906 may be provided for electrically connecting the multi-lead connector 908 to IPG 902 via a suitable connector interface or socket 903 that may be mated to an interface receptacle or header portion 905 of IMD 902. In general operation, electrical pulses may generated by the pulse generating circuitry 910 under the control of processing block 912, which may be provided to the switching circuitry 920 that is operative to selectively connect to the electrical outputs of the IMD, which are ultimately coupled to one or more electrodes of any combination of leads 904-1 to 904-M at a distal end of the lead system via respective electrical conductive traces.

An external device 930 may be implemented to charge/recharge the battery 918 of IMD 902, to access memory 914, and/or to program or reprogram IMD 902 with respect to the stimulation set parameters including pulsing specifications while implanted within the patient (although a separate recharging device could alternatively be employed). In alternative embodiments, accordingly, separate programmer and charger devices may be employed for charging and/or programming IMD 902 and/or any programmable components thereof. Regardless of whether charging functionalities and communication/programming functionalities are integrated, an example embodiment of the external device 930 may be a processor-based system that possesses wireline and/or wireless communication capabilities, near field magnetic/RF coupling capabilities, etc. Software may be stored within a non-transitory memory of the external device 930, which may be executed by the processor 936 to control the various operations of the external device 930. A connector or “wand” 934 may be electrically coupled to the external device 930 through suitable electrical connectors (not specifically shown), which may be electrically connected to a telemetry/charging component 932 (e.g., inductor coil, RF transceiver, etc.) at the distal end of wand 934 through respective links that allow bi-directional communication with IMD 902. Optionally, in some embodiments, wand 934 may comprise one or more temperature sensors for use during charging operations.

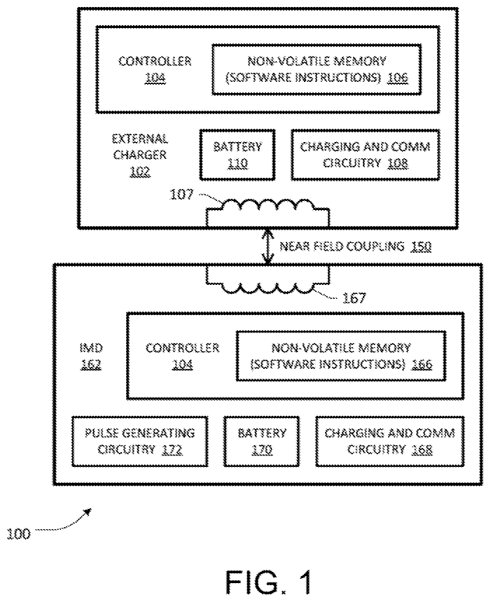

Turning attention now to FIG. 1, depicted therein is a block diagram of charging system 100 comprising an external charger 102 and an IPG device 162 that includes an embodiment of wireless power transfer circuitry according to the teachings herein. For purposes of the present patent disclosure, example IPG 162 may comprise any of the IMDs having any number or type of lead systems set forth above. Accordingly, the terms “IMD”, “IPG”, or related terms of similar import will be somewhat synonymously used herein. In one arrangement, charger 102 may include a controller or processor 104 (e.g., any suitable commercially available microcontroller) for controlling the operations of charger 102 according to instructions stored in non-volatile (non-transitory) memory 106. In one arrangement, charger 102 may be powered by a battery 110 having a suitable output voltage range. In some embodiments, battery 110 may comprise a rechargeable Lithium (Li) ion battery although other battery types or chemistries may be used. In some further embodiments, inductive step-up converters may be used in conjunction with a battery to obtain a suitable coil drive voltage. External charger 102 also comprises charging and communication circuitry 108, which may be adapted or otherwise configured in some embodiments to electrically couple to a coil 107 operating as a charging energy source. In some embodiments, coil 107 may be disposed in an external wand (not shown in this FIG.) that may be held, during charging, by a patient or an authorized healthcare professional about the patient's body adjacent to an implant site of IMD 162. Alternatively, the charger's coil 107 (which may be referred to as a primary coil) may be integrated in the same device package with the circuitry of charger 102. Preferably, charging and communication circuitry 108 may be configured to drive the primary coil 107 using a suitable RF signal for charging purposes. In some embodiments, charging and communication circuitry 108 may also drive the coil 107 using a suitable modulated RF signal to communicate/receive data to/from IMD 162. In still further embodiments, charger 102 may also be adapted for use as a controller to control the operations of IMD 162 by communicating suitable control parameters using communication circuitry 108, as noted above.

Example IMD 162, which is another representation of IMD 902 described above, is illustrated herein as comprising controller 164 (e.g., any suitable commercially available microcontroller that may be specially programmed for use as described herein) for controlling the pulse generation functionalities and other operations of IMD 162 according to instructions stored in non-volatile memory 166. IMD 162 comprises pulse generating circuitry 172 for generating stimulation pulses for delivery to tissue of the patient. It should be appreciated that any suitable existing or later developed pulse generating circuitry may be employed. An example of pulse generating circuitry is described in U.S. Patent Application Publication No. 2006/0259098, entitled “SYSTEMS AND METHODS FOR USE IN PULSE GENERATION,” which is incorporated herein by reference. Pulse generating circuitry 172 may comprise one or multiple pulse sources. Also, pulse generating circuitry 172 may operate according to constant voltage stimulation, constant current stimulation, or any other suitable mode of operation.

The various components of IMD 162 are powered by one or more internal batteries 170 (e.g., Li-ion rechargeable batteries, NiCad or the like). Battery 170 may be recharged by converting electromagnetic, such as RF, power radiated or received from external charger 102. Charging and communication circuitry 168 of IMD 162 is operative to couple to a coil 167 (referred to as a secondary coil) for effectuating near field coupling 150 with the coil 107 of external charger 102. When external charger 102 radiates RF power using its coil 107, the inductive coupling between the coil 107 of charger 102 with the coil 167 of IMD 162 causes an alternating current to be induced in the coil 167 of IMD 162. As will be set forth in detail further below, at least a portion of circuitry 168 may be configured to utilize the induced current in order to provide a charging voltage/current to battery 170 in a controllable manner. Also, in some embodiments, circuitry 168 may optionally use the same coil 167 to effectuate control communications signaling with charger 102. Further, it will be seen that an embodiment of the present disclosure advantageously uses only two feedthrough connections for connecting a coil-based frontend portion disposed in the header portion of IMD 162 to the rest of the internal circuitry of IMD 162. As skilled artisans will appreciate, the pulse generation circuitry 172 may be coupled to one or more stimulation leads through electrical connections provided in the header portion of the IMD's housing (i.e., feedthroughs), and by minimizing the number of feedthroughs used for connecting electrical conductors for other purposes (e.g., charging/communications), the number of leads that may be deployed in a stimulation therapy system may be advantageously maximized.

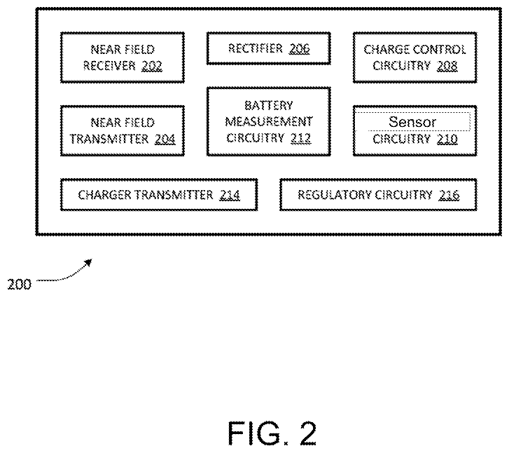

FIG. 2 depicts a block diagram of charging circuitry 200, which is a further representation of circuitry 168 of FIG. 1, illustrating additional components thereof according to one example embodiment. Circuitry 200 comprises coil and bridge rectifier circuitry 206, wherein a coil thereof (e.g., secondary coil 167 shown in FIG. 1) may be used for charging operations as well as communications with an external charger (e.g., charger 102) in some embodiments. In some other embodiments, the secondary coil may be used only for charging, with alterative links being available for communication purposes as previously noted. A near field receiver 202 is coupled to the coil, e.g., through a suitable capacitive arrangement as will be set forth further below. In one arrangement, receiver 202 may be configured to demodulate data when a carrier at an appropriate frequency is detected, whereupon a data stream may be communicated to controller 164. In similar fashion, near field transmitter 204 may be configured in one arrangement to receive a data stream from controller 164 for generating a modulated RF signal therefor, which may be applied to the secondary coil to communicate data via NFC to charger 102. Signal modulation and demodulation may, alternatively, be implemented in software executing on controller 164. Further, in some example embodiments, near field receiver 202 and transmitter 204 may be configured to not operate (e.g., disabled) when charging operations are taking place. Accordingly, a separate charger transmitter 214 may be employed to provide charging status messages to charger 102 when charging/discharging operations are being effectuated.

In one example arrangement, sensor circuitry 210 may be provided to measure certain aspects of the IMD, such as temperature of the device or an individual component thereof, such as the temperature of the battery 170, the metal can (housing) of the IMD, the coil 107, controller 104, voltage rectifier 206, or any other component of charging circuitry 200, for control of charging operations. In one embodiment, regulatory circuitry 216 is configured to control charging operations in response to one or more feedback/measurement signals (e.g., from sensor circuitry 210).

In one embodiment, charge control circuitry 208 may be provided to control the charging of battery 170. In one embodiment, charge control circuitry 208 may be configured to use the measurement functionality of battery measurement circuitry 212 to detect the state of battery 170. By way of illustration, charge control circuitry 208 may be operable to be battery measurement circuitry to measure the battery voltage, charging current, battery discharge current, temperature and/or the like. In some example embodiments, charge control circuitry 208 may prevent battery charging when an end-of-life (EOL) state has been reached for battery 170, which may be determined responsive to measurements provided by battery measurement circuitry 212. In further embodiments, charge control circuitry 208 may be configured to use a number of measurements to conduct fast charging operations as disclosed in greater detail in U.S. Patent Application Publication No. 2006/0259098, entitled “SYSTEMS AND METHODS FOR USE IN PULSE GENERATION,” incorporated by reference hereinabove. In still further embodiments, charge control circuitry 208 may also be configured to monitor one or more output signals from sensor circuitry 210 to further regulate the output voltage from rectifier circuitry 206.

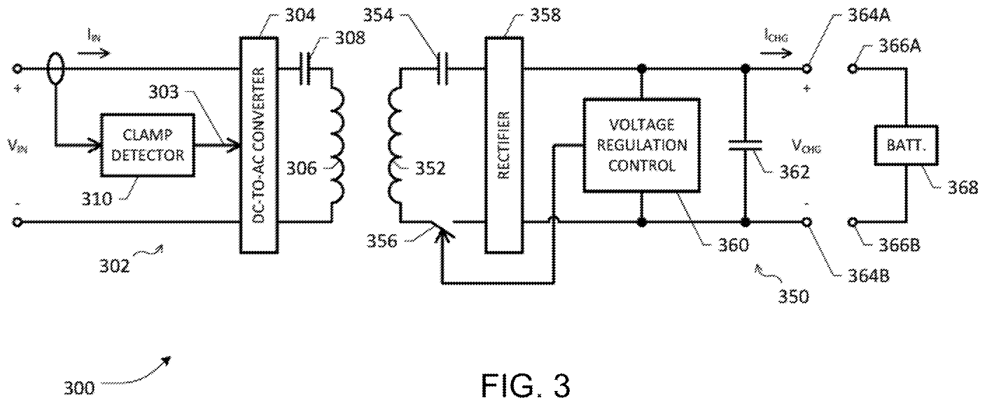

FIG. 3 is a high level circuit block diagram of a wireless charging system 300 for purposes of an example embodiment of the present disclosure. Broadly, a power sender block 302 is operative as an external charger 102 that supplies RF energy to a power receiver block 350 (e.g., an IMD 162, 900) through respective series resonant coils that operate as a loosely coupled transformer (i.e., via magnetic coupling). A DC voltage input (V1N) having a suitably configurable voltage range is provided to the power sender block 302, which includes a DC-to-AC converter 304 coupled to a sender-side tuning circuit comprising a primary coil 306 and a capacitor 308 connected in series. A clamp detector/monitor 310 may be included in the power sender block 302 for sensing the state of input current (11N). In one example embodiment, clamp detector/monitor 310 may be configured to generate a control signal 303 to DC-to-AC converter 304 in response to the input current status. It should be appreciated that DC-to-AC converter 304 is operative as a coil driver in order to supply adequate RF power to the power receiver block 350. When power receiver block 350 is not accepting power during a charging cycle (e.g., due to internal voltage/charging regulation and/or other internal ambient and status control signals), current flow through the sender-side tuning circuit is negligible (i.e., turned off), which condition may be sensed as a status change in the input current by the clamp detector/monitor circuitry 310 to generate control signal 303 operative to deactivate the power sender circuitry during the off state, thereby saving power.

To effectuate near field inductive RF power transfer, the power receiver block 350 is provided with a receiver-side tuning circuit comprising at least a secondary coil 352 coupled to at least a capacitor 354 in series (e.g., similar to the sender-side tuning circuit arrangement). An induced AC signal from the receiver-side tuning circuit is rectified by a rectifier 358, whose output may be optionally and/or suitably conditioned to apply power to a load, i.e., a battery 368 having terminals 366A, 3668. In an example arrangement, battery 368 may be disposed between output nodes 364A, 3648 of conditioning circuitry having an output capacitor arrangement (C_{OUT}) 362 for providing a suitable DC output voltage (V_{CHG} or V_{OUT}). In one example embodiment, voltage regulation control circuitry 360 may be coupled between the rectifier/conditioning portion 358 and battery load 368, which may be configured to generate one or more control signals for controlling a series switch arrangement 356 connected to the receiver-side tuning circuit arrangement.

It should be appreciated that the relationships between the sender-side coil voltage and current and the receiver-side coil voltage and current may be determined in an example implementation by the series tuning of the respective coils. For instance, such relationships may depend upon the operating frequency, tuning accuracy, coil separation, coil geometries, and the like. Accordingly, power transfer in an example arrangement involving wireless charging system 300 may in general depend on coupling between coils 306, 352, which in turn may depend on the distance between coils 306, 352, alignment, coil dimensions, coil materials, respective number of turns, magnetic shielding, impedance matching, applicable power band and associated resonant frequency, duty cycle, etc. Skilled artisans will recognize that at least some of these parameters may be selected in the design of an embodiment in order to comply with known or heretofore unknown wireless power transfer standards and specifications (e.g., Wireless Power Consortium WPC 1.1 Standard). Further, the voltage regulation control circuitry 360 may be appropriately configured in an example embodiment such that the time spent in the ON and OFF states may be suitably designed depending on the IMD application. In one embodiment, the time spent in the ON and OFF states is used as a machine readable code for indicating the temperature of one or more components of the IMD 162, as further described below.

In another embodiment, the time spent in the ON and OFF states may be determined based on an applicable voltage hysteresis band (V_{HIGH}−V_{LOW}), the rectifier output current IR and the load current Iₒᵤᵣ. In one example embodiment, an upper output threshold voltage V_{HIGH} that begins clamping may be selected to be at 4.5V and a lower threshold voltage V_{LOW} that ends clamping may be selected to be at 4.1 V, resulting in a nominal voltage hysteresis voltage of 0.13 V. In embodiments, the DC voltage should be within a range of from 4.1V to 4.5V. In an example embodiment, the power sender block 302 may be configured to continually adjust its RF output power to maintain at least substantially constant power transfer to the power receiver block 350 across a range of distances. Further, certain additional design criteria may be implemented in order to achieve maximum power transfer efficiency in an implementation. For example, one requirement may be that the charger, i.e., power sender block 302, should deliver a select battery charging current suitable for a use case or application scenario. In an example use case, such a requirement may comprise a charging current of 50 mA. Another design requirement may be that the charger should deactivate during the OFF states to conserve power.

Accordingly, in one arrangement, the clamp detector/monitor circuit 310 of the power sender block 302 may be configured to sense the time periods between clamping events of the power receiver block 350 in order to modulate the output power, as previously noted. Related details with respect to utilizing a clamp detection signal in a charging system may be found in U.S. Pat. No. 8,731,682, entitled “EXTERNAL CHARGING DEVICE FOR CHARGING AN IMPLANTABLE MEDICAL DEVICE AND METHODS OF REGULATING DUTY CYCLE OF AN EXTERNAL CHARGING DEVICE,” incorporated by herein.

In one embodiment, the wireless charging system 300 may be configured such that it involves only two feedthrough connections for connecting the receiver-side tuning circuit comprising coil 352 and capacitor 354 to the rest of the IMD internal circuitry. Moreover, the series switch arrangement 356 may be configured such that the receiver-side tuning circuit may be detuned or otherwise disabled during the OFF condition, thereby advantageously eliminating a high voltage condition that can develop during the time when the power receiver block 350 is in the clamped state because the receiver-side tuning circuit may be in resonance. As one skilled in the art will appreciate, the voltage in the secondary coil 352 can reach significantly high levels in the clamped state in some implementations (e.g., as high as 300V), which is highly undesirable in an IMD application. It is noted that the secondary coil 352 may be the same as secondary coil 167 in some embodiments.

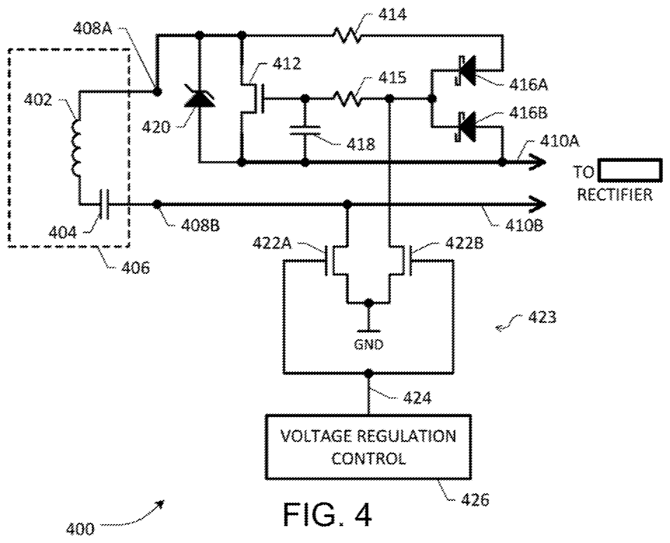

FIG. 4 depicts a circuit diagram of a frontend portion 400 of a rechargeable IMD/IPG device operating as a power receiver for facilitating wireless power transfer according to an example embodiment of the present disclosure. An inductive coupling element 406 comprising at least one inductor or coil 402 connected with at least one capacitor 404 in a series LC circuit configuration is operative as a receiver-side tuning circuit wherein the at least one capacitor 404 may be configured to be tunable over a range of frequencies. In one example implementation, coil 402 may comprise an inductor or its equivalent having an inductance of about 350-500 microhenries (pH) and tuning capacitor 404 may comprise a capacitance of about 500-1000 picofarads (pF) or its equivalent. Regardless of the actual number and/or type of inductors and/or tuning capacitors used in a particular implementation, a lumped-element model of the series LC circuit configuration of RF coupling element 406 may preferably be connected in an arrangement that defines a first electrical node 408A at a terminal of at least one inductor 402 and a second electrical node 4088 at a terminal of at least one capacitor 404. Where the LC circuit configuration forming the inductive coupling element 406 is disposed in the IMD's header, nodes 408A/408B are operative to be electrically connected to the remainder of the frontend circuitry 400 via respective feedthroughs in accordance with the teachings herein. A series switch 412 is disposed between the first electrical node 408A and a trace 410A coupled to an input terminal of a bridge rectifier (not shown in this FIG.) for detuning the LC circuit element 406 during the OFF state of the power receiver. In one embodiment, switch 412 may comprise an N-channel metal oxide semiconductor field-effect transistor (NMOSFET) that may be opened when the charging is OFF. During the ON period, switch 412 may be configured to be automatically closed by deriving a gate drive voltage from the LC circuit element 406. On the other hand, switch 412 may be configured to be opened in the OFF state responsive to a gate control signal derived from a clamp signal using appropriate logic circuitry. Skilled artisans will recognize upon reference hereto that suitable switch protection circuitry and/or ON-state gate control circuitry may be provided using appropriate electrical/electronic components including but not limited to, inter alia, capacitors, transistors, FETs, diodes, etc., in various combinations to control and condition power transfer operations depending on a particular wireless power transfer application.

In one implementation, a Zener diode 420 may be connected between drain and source nodes/terminals of switch 412 in order to provide protection therefor against inductive spikes. For example, a Zener diode of appropriate electrical characteristics may be disposed for providing clamping protection against inductive spikes at around 30 V to 60 V. A pair of Schottky diodes 416A, 416B coupled in a configuration such that respective cathodes thereof are commonly connected to a resistor 415, which in turn is connected to a gate of switch FET 412. A capacitor 418 may be disposed between the gate and one of the terminals of switch FET 412 (e.g., source node coupled to bridge rectifier trace 410A). Anode terminal of Schottky diode 416A is coupled to a resistor 414, which in turn is commonly connected to the cathode terminal of Zener diode 420, first electrical node 408A and a terminal of switch FET 412 (e.g., drain). On the other hand, anode terminal of Schottky diode 416B may be directly coupled to bridge rectifier trace 410A. In one implementation, resistor 414 may have a resistance of about 5-15 kOhms and resistor 415 may have a resistance of about 0.5-1.5 kOhms. In one implementation, capacitor 418 may comprise a capacitor rated to about 50 V±10% and having a capacitance of about 500-1500 pF.

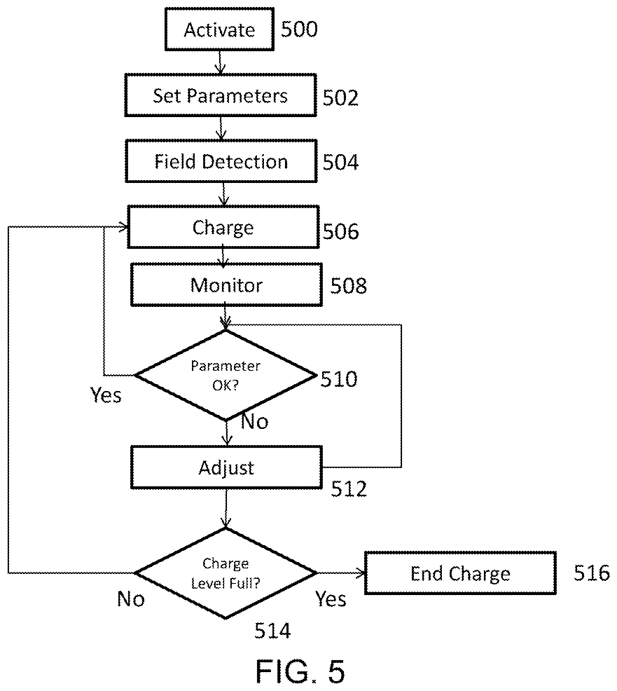

Appropriate control signaling for the LC circuit configuration of inductive coupling element 406 and as well as gate control for switch 412 may be effectuated by way of a frontend control signaling portion 423 (referred to herein as a “clamp circuit” or “clamp control circuitry”) that is driven by a clamp control signal 424 generated by a voltage regulation control block 426 operative to provide clamping that in one embodiment is used to provide temperature data, as well as optional over-voltage protection in another embodiment. In one implementation, clamp control signaling portion 423 comprises a pair of FETs 422A/4228 whose respective gates are driven by clamp control signal 424, wherein source terminals thereof are commonly tied to a reference potential, e.g., ground. Whereas a drain terminal of FET 4228 is connected to the common cathode connection of Schottky diodes 416A, 416B, a drain terminal of FET 422A is connected the second electrical node 408B formed at a terminal of at least one capacitor 404 of the LC circuit configuration. Further, the second electrical node 408B is also coupled to a trace 410B extending to a second input terminal of the bridge rectifier (shown in FIG. 5). In one implementation, when clamp control signal 424 is asserted (e.g., a logic high) during the OFF state, gate voltages of FETs 422A and 422B are driven high, thereby causing FETs 422A and 422B to be turned on. As FET 422A is turned on, the second electrical node 408B connected to bridge rectifier trace 410B is pulled to ground. At the same time, as FET 422B is turned on, it causes the gate terminal of series switch FET 412 to be pulled to ground. Accordingly, series switch FET 412 is turned off, thereby opening the series connection path between the first electrical node 408A and bridge rectifier trace 410A. As a result, the series LC circuit is opened during the OFF state, whereby it is caused to be detuned with respect to a primary coil in the external charger. Since it is detuned, there is no resonance-caused high voltage condition developed in the receiver-side circuitry of an IMD. As noted above, series switch FET 412 is automatically closed in the ON state (e.g., clamp control signal 424 is deasserted), wherein a suitable gate drive voltage is derived from the LC circuit component 406 whose output is conditioned through the Schottky diode arrangement 416A/416B.

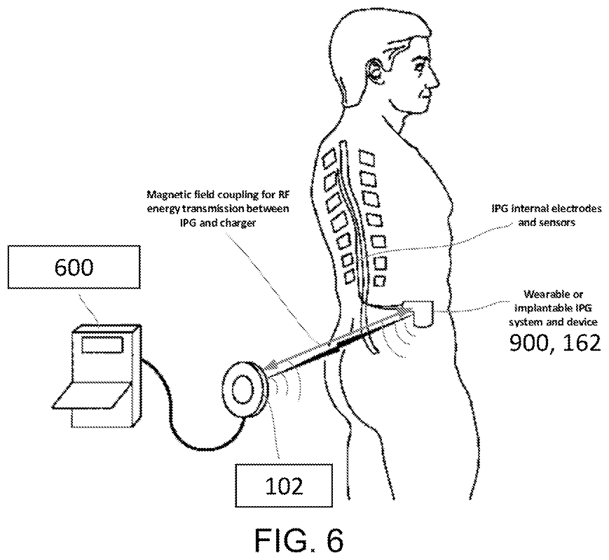

A process for charging an IMD according to the present disclosure is now further described with reference to FIG. 5. At operation 500, the external charger 102 is activated, for example by pressing a button or powering on the charger 102 using a user interface 600 (FIG. 6). The user interface is electronically coupled to charger 102 and may include one or more of a display, such as an electronic display using LCD, LED or the like, and a user input, such as one or more buttons, keyboard, or touchscreen (which may be the display). After the external charger 102 has been activated, the charging parameters are set at operation 502. During the setting of parameters 502, one or more of the charging frequency, maximum current, minimum current, normal current and the like may be set for the charger 102. At this point, the charger 102 should be within close enough proximity to IMD 162 to enable the electric charging field (e.g., RF emissions) of external charger 102 to be received by coil 167. At operation 504, the charger 102 detects, using charging and comm circuitry 108, whether coil 107 is electromagnetically coupled to secondary coil 167 of the IMD 162. In another embodiment, the IMD 162, using charging and comm circuitry 168 detects whether coil 107 is electromagnetically coupled to secondary coil 167 of the IMD 162. Once such electromagnetic coupling of coil 107 to secondary coil 167 has occurred, mutual stabilization of the magnetic charging is facilitated, and the RF field emitted from coil 107 is received by secondary coil 167 such that a current is generated by coil 167 converted to a DC current by charging and comms circuitry 168 for charging battery 170.

Subsequently, a charging cycle 506 may begin. During the charging cycle 506 the typical converted DC voltage V_{CHG} (e.g., from the rectifier 358) is from about 4.1V to 4.5V, but may be any voltage that allows the charger to function as described herein. The converted DC voltage is used both to charge the battery 368 (which may be the same as battery 170) as well as the capacitor 362. The charging cycle 506 may cause the temperature of the IMD 162 to rise. However, in some embodiments, one or more of the IMD housing, coil 167, battery 368, secondary coil 352, charging and comm circuitry 168 or any other component of IMD 162 may rise during the charging cycle 506. The temperature of one or more of the components is measured by a temperature sensor of sensor circuitry 210.

Just prior to the beginning of the charging cycle 506, the temperature of the IMD 162 or one or more of its components is approximately within the range of from 36° C. to about 37° C. Once the charging cycle 506 begins, the temperature of the IMD 162 or one or more of its components starts to increase to within the range of from 37° C. to about 39° C. In one embodiment, the sensor circuitry outputs temperature data to the charge control circuitry 208. In one embodiment, the voltage regulation control block 426 utilizes the temperature data, which is received by the voltage regulation control block 426 to clamp the circuit, such that no load (OFF state) or no charging of the battery 368 occurs. Such clamping of the circuit, and the associated time spent in the ON and OFF states may be controlled using pulse width modulation (PWM) or semi-PWM controls, as further described herein. Such control of the clamping of the circuit facilitates controlling of the heat and temperature ramping rate of one or more components of the IMD.

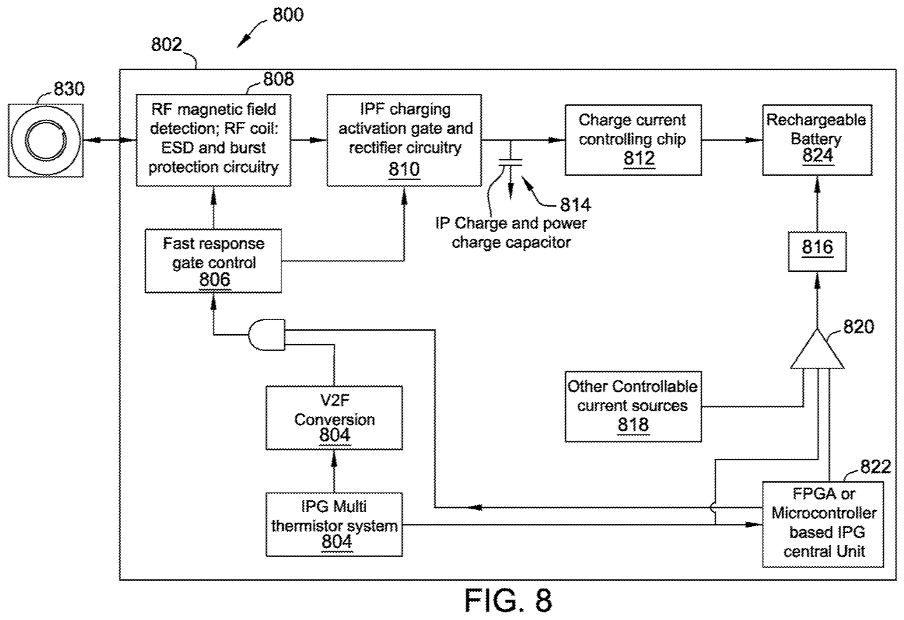

With reference to FIG. 8, an embodiment of the IMD with circuitry for controlling the charging procedure is further described. In one embodiment, an IMD 800 (which may be the same or similar to IMD 902, 162) includes a housing 802 (e.g., a metal body) for housing the IMD 800 componentry. Within the housing 802, there is housed one or more of the following components that are electronically coupled: a voltage to frequency (“V2F”) conversion circuit 804, a fast response gate control circuit 806, an RF circuit 808, a rectifier circuit 810, a charge control circuit 812, a power store capacitor 814, a constant current source 816, a controllable current source 818, an integrated circuit 820, and a central processor 822, a battery 824 and a temperature sensor system 826. An external charger 830 is placed within close proximity to the IMD 802, such that charging power from the external charger 830 is wirelessly transmitted to the RF circuit 808 during charging. The external charger 830 may be the same or similar to external charger 102 described above.

In one embodiment, during a charging procedure, the external charger 830 outputs RF power, such as via coil 107 (FIG. 1). The RF power may be outputted in some embodiments within a frequency range of from about 280 kHz to about 320 kHz. The coil 107 outputs the RF power to be received at RF circuit 808, which may include a secondary coil, such as secondary coil 167, which subsequently generates a charging current as described herein for charging the battery 824.

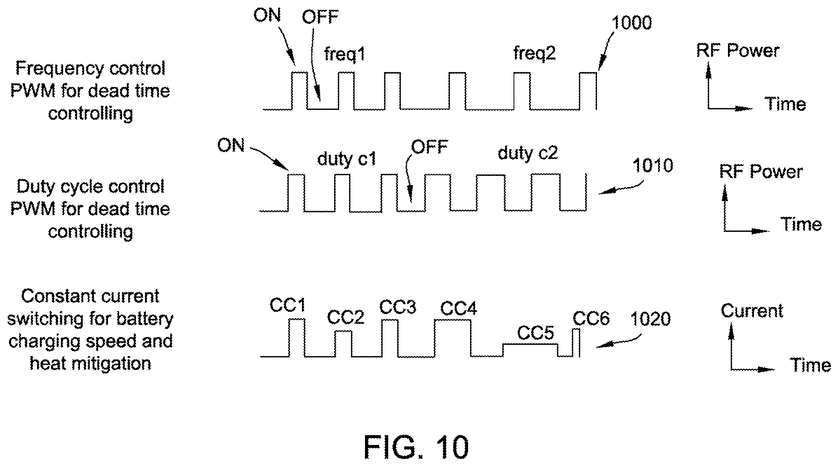

In one embodiment, the RF power output by the coil 107 is correlated to a PWM signal cycle time that is synchronized with thermistor output signals from the thermistor system 826. In one embodiment, the temperature sensor system 804 includes one or more temperature sensors, such as thermistors, that detect a temperature of one or more components of the IMD, such as housing 802, coil 107 (FIG. 1), RF circuit 808, battery 824 or any other component housed within housing 802. The temperature sensor system 826 outputs a temperature signal representative of the temperature of the monitored component. In one embodiment, the temperature signal is electronically communicated to the V2F conversion circuit to be converted to a frequency based signal. The signal is received at the RF circuit 808, in one embodiment via the fast response gate control circuit 806. Upon receiving the frequency based signal, the RF circuit 808 outputs a waveform 1000 (FIG. 10) that is received by the external charger 830 and which communicates temperature data to the external charger 830.

External charger 830 is subsequently controlled to control the RF power output activation based upon the received waveform 1000. For example, freq1 may represent a normal (non-temperature regulated) charging state, wherein the charging RF output is controlled to follow a predetermined, or scheduled, amount of time in the ON state in which RF power is being output by external charger 830. The ON state is represented in freq1 by the upper horizontal portion of the line, while the OFF state is represented by the troughs. In the normal charging state, the RF power output by the external charger is matched to output power according to the received waveform freq1. During temperature regulated charging, such as when the temperature of a monitored component meets or exceeds a predetermined threshold, the RF circuit 808 outputs a modified waveform freq2, which has a different frequency PWM than freq1. For example, if the temperature of a monitored component meets or exceeds a predetermined threshold that requires a 50% duty cycle to allow sufficient cooling time for the temperature measured IMD component, the ON state in the freq2 waveform occurs only 50% as often as in the freq1 waveform, (i.e., the frequency of the ON state in freq2 is half the frequency of the ON state in freq1). It should be noted that the freq2 waveform is controlled such that the frequency of the ON and OFF states is variable to denote an appropriate RF power duty cycle to allow sufficient cooling of the component, such as from 0% to 100% of the frequency of freq1. For example, higher temperatures of the IMD components may require additional time i the OFF state, while lower temperatures may allow for additional time in the ON state (e.g., less time in the OFF state). In this embodiment, the amplitude of the waveform 1000 in the ON state in freq1 and freq2 may be substantially the same during each ON cycle.

In another embodiment, external charger 830 is operated in a manner to control the RF power output activation based upon the received waveform 1010. For example, in this embodiment duty c2 may represent a normal (non-temperature regulated) charging state, wherein the charging RF output is controlled to follow a predetermined, or scheduled, duty cycle in the ON state in which RF power is being output by external charger 830. The ON state is represented in waveform 1010 at duty c1 by the upper horizontal portion of the line, while the OFF state is represented by the troughs. In the normal charging state duty c2, the RF power output by the external charger is matched to output power according to the received waveform duty c2. During temperature regulated charging, such as when the temperature of a monitored component meets or exceeds a predetermined threshold, the RF circuit 808 outputs a modified waveform duty c1, which has a different duty cycle PWM than duty c2. In this embodiment, the frequency at which the ON state occurs in duty c2 is the same frequency at which the ON state occurs in duty c1. However, for example, if the temperature of a monitored component meets or exceeds a predetermined threshold that requires a 50% duty cycle to allow sufficient cooling time for the temperature measured IMD component, the ON state in the duty c1 waveform occurs only 50% as long of a time as in the duty c2 waveform. It should be noted that the duty c1 waveform is controlled such that the length of time in the ON and OFF states is variable to denote an appropriate RF power duty cycle to allow sufficient cooling of the component, such as from 0% to 100% of the duty cycle time of duty c2. For example, higher temperatures of the IMD components may require additional time i the OFF state, while lower temperatures may allow for additional time in the ON state (e.g., less time in the OFF state). In this embodiment, the amplitude of the waveform 1010 in the ON state in duty c1 and duty c2 may be substantially the same during each ON cycle.

In another embodiment, external charger 830 is operated in a manner to control the RF output current based upon the received waveform 1020. For example, in this embodiment the waveform 1020 represents constant current switching levels. In this embodiment, the charging RF output current is controlled to continuously adjust to facilitate heat mitigation and desired charging speed. For waveform 1020, the ON state in which RF output current is being output by external charger 830. The ON state is represented in waveform 1020 by the upper horizontal portion of the line, while the OFF state is represented by the troughs. In this embodiment, the amplitude of the RF output current is denoted by the vertical magnitude of each peak (i.e., a taller vertical peak indicates a greater output current, while the duration of time the given current is output is represented horizontally such that a longer horizontal period indicates a longer time period. In this embodiment, the RF power output by the external charger is matched to output power according to the received waveform 1020. Accordingly, waveform 1020 is used by external charger 830 to adjust its output current to each ON and OFF state of cc1-cc6. It is noted that although waveform 1020 is shown with six peaks, the waveform may have any number of peaks to allow the device to function as described herein. In the waveform shown, each peak cc1-cc6 may be based upon a temperature of a component detected by a temperature sensor system 804. For example, cc1 may represent a current pulse (amplitude and time) during a normal non-temperature regulated charging state. Accordingly, cc2, having a reduced amplitude may be used if an increase in temperature is detected by temperature sensor system 804. Accordingly, the reduced current cycle of cc2 will generate less heat during charging than cc1, thus mitigating heat effects. Accordingly, the peaks of waveform 1020 are adjusted to facilitate heat mitigation and charging speed. If the temperature detected by temperature sensor system 804 is low, the amplitude or time of the current pulse may be adjusted to be higher amplitude or longer time period to more rapidly charge the battery when heat effects are not problematic. In some embodiments, higher temperatures of the IMD components may require additional time in the OFF state or lower amplitude of the current, while lower temperatures may allow for additional time in the ON state (e.g., less time in the OFF state) or higher amplitude of the current.

In another embodiment, the output power/current of the external charger 830 is maintained at a constant level and the charge control circuit 812 is operated in a manner to control the RF power or current output based upon the received waveform 1000, 1010 or 1020. Accordingly, in this embodiment, the waveform 1000, 1010 or 1020 is received from the temperature sensor system 804 at the charge current controlling circuit 812. In some embodiments, the waveform is first processed by one or more of the V2F circuit 804 and or the central processing unit 822 prior to being communicated to the charge current controlling circuit 812. In this embodiment, the output current that is directed to rechargeable battery 824 is adjusted by charge current control circuit 812 based upon the received waveform 1000, 1010 or 1020 to mitigate heat and/or to adjust desired charging speed.

In embodiments, the rechargeable battery 824 may be charged at different current levels based on desired charging speed. In one embodiment, for rapid charging, sometimes referred to as super-charging, the current is controlled using one or more of the waveforms 1000, 1010 or 1020 and processes and devices described hereinabove, to be a current of from 75 mA to 1000 mA as the recharge current. As used herein, the recharge current is the current level received at the rechargeable battery 824. In another embodiment, during normal (non-temperature adjusted charging), the recharge current is controlled to be from 30 mA to 50 mA. In other embodiments, during temperature restricted charging, the recharge current is controlled to be from 10 mA to less than 30 mA.

In embodiments, if the determined temperature parameter (e.g., as detected by temperature sensor circuit 804) falls within a predetermined threshold, such as from 38° C. to 39° C., (e.g., low risk) the output power of the power/current of external charger 830, or charge current controller 812 may be maintained at a specified value, such as a normal (non-temperature adjusted) charging level. However, if the determined temperature parameter falls within a predetermined threshold indicated to be of increased risk (such as a medium risk or high risk) based upon the determined temperature, such as any temperature above 39° C., power/current of external charger 830, or charge current controller 812 may be adjusted 512. For example, if the determined temperature presents an increased risk, one or more of the power/current of external charger 830, or charge current controller 812 may be reduced until the determined temperature value is reduced to a within the predetermined threshold or below.

At operation 514, the level of charge of the battery 824 is determined by the charger 102. The level of charge may be determined, in some embodiments, by using the determined temperature level of the battery 824. In other embodiments, the IMD 162 may send a signal via charging and comm circuitry 168 of the IMD 162 to the charging and comm circuitry 108 of the charger 102 indicating a charge level of the battery. If the charge level of the battery 824 is full, the external charger 830 is controlled to turn off to end the charging cycle. However, if the charging level is indicated to be less than full, the charging cycle 506 is continued.

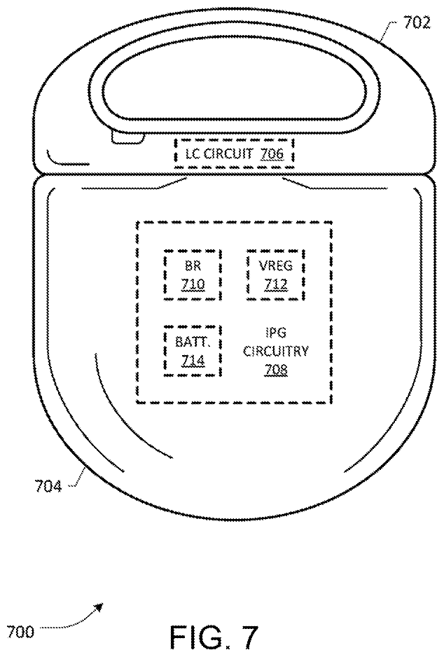

FIG. 7 depicts an example IMD/IPG housing 700 having a header portion 702 and a body portion 704 wherein an embodiment of the present disclosure may be practiced. Regardless of any particular form factor, header portion 702 may preferably be configured to operate as a housing portion for an inductive coupling component or circuit that may comprise one or more inductors and one or more tuning capacitors in a series LC configuration 706 having two feedthrough terminals. Likewise, body portion 704 may be configured to house an IPG circuit portion 708 that may include various pieces of the circuitry described in detail hereinabove, e.g., including frontend circuitry portion, bridge circuitry portion, voltage regulation circuitry portion, battery, etc., as exemplified by various blocks 710, 712, 714, in addition to one or more other blocks or functionalities set forth in reference to FIG. 9. As previously noted, electrical connectivity between LC configuration circuit 706 and IPG circuit portion 708 may be accomplished using only two feedthrough paths controlled by a series detuning switch in accordance with the described above, whereby the availability of remaining feedthroughs may be maximized for other purposes (e.g., for supporting additional lead systems).

In the above-description of various embodiments of the present disclosure, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and may not be interpreted in an idealized or overly formal sense expressly so defined herein.

At least some example embodiments are described herein with reference to one or more circuit diagrams/schematics, block diagrams and/or flowchart illustrations. It is understood that such diagrams and/or flowchart illustrations, and combinations of blocks in the block diagrams and/or flowchart illustrations, can be implemented by any appropriate circuitry configured to achieve the desired functionalities. Accordingly, example embodiments of the present disclosure may be embodied in hardware and/or in software (including firmware, resident software, micro-code, etc.) operating in conjunction with suitable processing units or microcontrollers, which may collectively be referred to as “circuitry,” “a module” or variants thereof. An example processing unit or a module may include, by way of illustration, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGA) circuits, any other type of integrated circuit (IC), and/or a state machine, as well as programmable system devices (PSDs) employing system-on-chip (SoC) architectures that combine memory functions with programmable logic on a chip that is designed to work with a standard microcontroller. Example memory modules or storage circuitry may include volatile and/or nonvolatile memories such as, e.g., random access memory (RAM), electrically erasable/programmable read-only memories (EEPROMs) or UV-EPROMS, one-time programmable (OTP) memories, Flash memories, static RAM (SRAM), etc.

Skilled artisans will recognize upon reference hereto that various switching components of one or more circuits described herein may be implemented using a variety of monolithic or integrated semiconductor devices known in the electrical arts, e.g., including but not limited to bipolar junction transistors (BJTs), metal oxide semiconductor field effect transistors (MOSFETS), junction gate FETs (JFETs), n-channel MOSFET (NMOS) devices, p-channel MOSFET (PMOS) devices, depletion-mode or enhancement-mode devices, and the like, as well as any logic gates built therefrom. Likewise, various types of comparators, e.g., inverting and/or non-inverting comparators, latched comparators, single ended comparators, differential op amp circuits and the like may be implemented in an example embodiment. It will be further understood that the sizing (e.g., channel width and length) and biasing of the switching devices used in any of the components can be highly configurable, depending on the voltage/current ratings, application requirements, and the like.

Further, in at least some additional and/or alternative implementations, the functions/acts described in the blocks may occur out of the order shown in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Furthermore, although some of the diagrams include arrows on communication paths to show a primary direction of communication, it is to be understood that communication may occur in the opposite direction relative to the depicted arrows. Finally, other blocks may be added/inserted between the blocks that are illustrated.

It should therefore be clearly understood that the order or sequence of the acts, steps, functions, components or blocks illustrated in any of the flowcharts depicted in the drawing Figures of the present disclosure may be modified, altered, replaced, customized or otherwise rearranged within a particular flowchart, including deletion or omission of a particular act, step, function, component or block. Moreover, the acts, steps, functions, components or blocks illustrated in a particular flowchart may be inter-mixed or otherwise inter-arranged or rearranged with the acts, steps, functions, components or blocks illustrated in another flowchart in order to effectuate additional variations, modifications and configurations with respect to one or more processes for purposes of practicing the teachings of the present patent disclosure.

The following embodiments are provided to illustrate aspects of the disclosure, although the embodiments are not intended to be limiting and other aspects and/or embodiments may also be provided.

Embodiment 1. An implantable medical device (IMD) configured to provide stimulation therapy to a patient, the IMD comprising: a rechargeable battery; pulse generating circuitry powered by the rechargeable battery; an inductive coupling element including at least one inductor operative to accept radio frequency (RF) power from an external charger and generate a charging voltage, a temperature sensor configured to measure a temperature of at least a part of the IMD and output measured temperature data; a waveform generating circuit for converting measured temperature data to a waveform signal for controlling a recharging current for the rechargeable battery; and wherein the inductive coupling element is configured to communicate the waveform signal to the external charger.

Embodiment 2. The IMD as recited in Embodiment 1, wherein the waveform signal is in the form of a frequency controlled pulse width modulation waveform.

Embodiment 3. The IMD as recited in any previous Embodiment, wherein the waveform generating circuit is configured to reduce a frequency of ON cycles if the measured temperature data meets or exceeds a predefined threshold.

Embodiment 4. The IMD as recited in any previous Embodiment, wherein the waveform signal is in the form of a duty cycle controlled pulse width modulation waveform.

Embodiment 5. The IMD as recited in any previous Embodiment, wherein the waveform generating circuit is configured to reduce a duty cycle time of ON cycles if the measured temperature data meets or exceeds a predefined threshold.

Embodiment 6. The IMD as recited in any previous Embodiment, wherein the waveform signal is in the form of a constant current switching waveform.

Embodiment 7. The IMD as recited in any previous Embodiment, wherein the waveform generating circuit is configured to reduce one or more of an amplitude of ON cycles or a length of time of the ON cycles if the measured temperature data meets or exceeds a predefined threshold.

Embodiment 8. The IMD as recited in any previous Embodiment, wherein the temperature sensor measures the temperature of at least one of the battery, a housing of the IMD, the inductive coupling element or the pulse generating circuitry.

Embodiment 9. A charging system for an implantable medical device, the system comprising: an external charging unit configured to provide radio frequency (RF) power; an IMD comprising: a rechargeable battery; pulse generating circuitry powered by the rechargeable battery; an inductive coupling element including at least one inductor operative to accept the radio frequency (RF) power from the external charger and generate a charging voltage, a temperature sensor configured to measure a temperature of at least a part of the IMD and output measured temperature data; a waveform generating circuit for converting measured temperature data to a waveform signal for controlling a recharging current for the rechargeable battery; and wherein the inductive coupling element is configured to communicate the waveform signal to the external charger, and the external charger provides the RF power based upon the waveform signal.

Embodiment 10. The system as recited in Embodiment 9, wherein the waveform signal is in the form of a frequency controlled pulse width modulation waveform.

Embodiment 11. The system as recited in any previous Embodiment, wherein the waveform generating circuit is configured to reduce a frequency of ON cycles if the measured temperature data meets or exceeds a predefined threshold.

Embodiment 12. The system as recited in any previous Embodiment, wherein the waveform signal is in the form of a duty cycle controlled pulse width modulation waveform.

Embodiment 13. The system as recited in any previous Embodiment, wherein the waveform generating circuit is configured to reduce a duty cycle time of ON cycles if the measured temperature data meets or exceeds a predefined threshold.

Embodiment 14. The system as recited in any previous Embodiment, wherein the waveform signal is in the form of a constant current switching waveform.

Embodiment 15. The system as recited in any previous Embodiment, wherein the waveform generating circuit is configured to reduce one or more of an amplitude of ON cycles or a length of time of the ON cycles if the measured temperature data meets or exceeds a predefined threshold.

Embodiment 16. The system as recited in any previous Embodiment, wherein the temperature sensor measures the temperature of at least one of the battery, a housing of the IMD, the inductive coupling element or the pulse generating circuitry.

Embodiment 17. A method of charging an implantable medical device (IMD), the IMD comprising a rechargeable battery, an inductive coupling element including at least one inductor operative to accept radio frequency (RF) power from an external charger and generate a charging current, the method comprising: measuring, using a temperature sensor housed within the IMD, a temperature of at least a part of the IMD and outputting measured temperature data; communicate the outputted measured temperature data to a waveform generating circuit within the IMD, and converting the measured temperature data to a waveform signal for controlling a recharging current for the rechargeable battery; transmitting the waveform signal to the external charger, and outputting RF power from the external charger based upon the waveform signal.

Embodiment 18. The method according to Embodiment 17, wherein the measured temperature data is converted to a frequency controlled pulse width modulation waveform.

Embodiment 19. The method according to any previous Embodiment, wherein converting the temperature data to the waveform signal comprises reducing a frequency of ON cycles if the measured temperature data meets or exceeds a predefined threshold.

Embodiment 20. The method according to any previous Embodiment, wherein the measured temperature data is converted to a duty cycle controlled pulse width modulation waveform.

Embodiment 21. The method according to any previous Embodiment, wherein converting the temperature data to the waveform signal comprises reducing a duty cycle time of ON cycles if the measured temperature data meets or exceeds a predefined threshold.

Embodiment 22. The method according to any previous Embodiment, wherein the measured temperature data is converted to a constant current switching waveform.

Embodiment 23. The method according to any previous Embodiment, wherein converting the temperature data to the waveform signal comprises reducing one or more of an amplitude of ON cycles or a length of time of the ON cycles if the measured temperature data meets or exceeds a predefined threshold.

Embodiment 24. The method according to any previous Embodiment, wherein measuring the temperature comprises measuring the temperature of at least one of the battery, a housing of the IMD, the inductive coupling element or the pulse generating circuitry.

Although various embodiments have been shown and described in detail, the claims are not limited to any particular embodiment or example. None of the above Detailed Description should be read as implying that any particular component, element, step, act, or function is essential such that it must be included in the scope of the claims. Reference to an element in the singular is not intended to mean “one and only one” unless explicitly so stated, but rather “one or more.” All structural and functional equivalents to the elements of the above-described embodiments that are known to those of ordinary skill in the art are expressly incorporated herein by reference and are intended to be encompassed by the present claims. Accordingly, those skilled in the art will recognize that the exemplary embodiments described herein can be practiced with various modifications and alterations within the spirit and scope of the claims appended below.

What is claimed is:

1. An implantable medical device (IMD) configured to provide stimulation therapy to a patient, the IMD comprising:
a rechargeable battery;
pulse generating circuitry powered by the rechargeable battery;
an inductive coupling element including at least one inductor operative to accept radio frequency (RF) power from an external charger and generate a charging voltage,
a temperature sensor configured to measure a temperature of at least a part of the IMD and output measured temperature data;
a waveform generating circuit for converting measured temperature data to a waveform signal for controlling a recharging current for the rechargeable battery; and
wherein the inductive coupling element is configured to communicate the waveform signal to the external charger.

2. The IMD as recited in claim 1, wherein the waveform signal is in the form of a frequency controlled pulse width modulation waveform.

3. The IMD as recited in claim 2, wherein the waveform generating circuit is configured to reduce a frequency of ON cycles if the measured temperature data meets or exceeds a predefined threshold.

4. The IMD as recited in claim 1, wherein the waveform signal is in the form of a duty cycle controlled pulse width modulation waveform.

5. The IMD as recited in claim 4, wherein the waveform generating circuit is configured to reduce a duty cycle time of ON cycles if the measured temperature data meets or exceeds a predefined threshold.

6. The IMD as recited in claim 1, wherein the waveform signal is in the form of a constant current switching waveform.

7. The IMD as recited in claim 6, wherein the waveform generating circuit is configured to reduce one or more of an amplitude of ON cycles or a length of time of the ON cycles if the measured temperature data meets or exceeds a predefined threshold.

8. The IMD as recited in claim 1, wherein the temperature sensor measures the temperature of at least one of the battery, a housing of the IMD, the inductive coupling element or the pulse generating circuitry.

9. A charging system for an implantable medical device, the system comprising:
an external charging unit configured to provide radio frequency (RF) power;
an IMD comprising:
a rechargeable battery;
pulse generating circuitry powered by the rechargeable battery;
an inductive coupling element including at least one inductor operative to accept t h e radio frequency (RF) power from the external charger and generate a charging voltage,
a temperature sensor configured to measure a temperature of at least a part of the IMD and output measured temperature data;
a waveform generating circuit for converting measured temperature data to a waveform signal for controlling a recharging current for the rechargeable battery; and
wherein the inductive coupling element is configured to communicate the waveform signal to the external charger, and the external charger provides the RF power based upon the waveform signal.

10. The system as recited in claim 9, wherein the waveform signal is in the form of a frequency controlled pulse width modulation waveform.

11. The system as recited in claim 10, wherein the waveform generating circuit is configured to reduce a frequency of ON cycles if the measured temperature data meets or exceeds a predefined threshold.

12. The system as recited in claim 9, wherein the waveform signal is in the form of a duty cycle controlled pulse width modulation waveform.

13. The system as recited in claim 12, wherein the waveform generating circuit is configured to reduce a duty cycle time of ON cycles if the measured temperature data meets or exceeds a predefined threshold.

14. The system as recited in claim 9, wherein the waveform signal is in the form of a constant current switching waveform.

15. The system as recited in claim 14, wherein the waveform generating circuit is configured to reduce one or more of an amplitude of ON cycles or a length of time of the ON cycles if the measured temperature data meets or exceeds a predefined threshold.

16. The system as recited in claim 9, wherein the temperature sensor measures the temperature of at least one of the battery, a housing of the IMD, the inductive coupling element or the pulse generating circuitry.

17. A method of charging an implantable medical device (IMD), the IMD comprising a rechargeable battery, an inductive coupling element including at least one inductor operative to accept radio frequency (RF) power from an
external charger and generate a charging current, the method comprising:
measuring, using a temperature sensor housed within the IMD, a temperature of at least a part of the IMD and outputting measured temperature data;
communicate the outputted measured temperature data to a waveform generating circuit within the IMD, and converting the measured temperature data to a waveform signal for controlling a recharging current for the rechargeable battery;
transmitting the waveform signal to the external charger, and
outputting RF power from the external charger based upon the waveform signal.

18. The method according to claim 17, wherein the measured temperature data is converted to a frequency controlled pulse width modulation waveform.

19. The method as recited in claim 17, wherein the measured temperature data is converted to a duty cycle controlled pulse width modulation waveform.

20. The method as recited in claim 17, wherein the measured temperature data is converted to a constant current switching waveform.
